(19) United States
(12) Patent Application Publication
(10) Pub. No.: US 2022/0323314 A1
(43) Pub. Date: Oct. 13, 2022

(54) DISSOLVABLE SOLID FOAM COMPOSITIONS

(71) Applicant: Plus Products, Inc., New York, NY (US)

(72) Inventors: Stephen Farias, Baltimore, MD (US); Scott Richard Gaboury, Blue Bell, PA (US); David Lee, Seoul (KR)

(21) Appl. No.: 17/712,525

(22) Filed: Apr. 4, 2022

Related U.S. Application Data
(60) Provisional application No. 63/170,815, filed on Apr. 5, 2021.

(51) Int. Cl. A61K 8/04; A61K 8/81; A61Q 19/10; A61Q 19/00

(52) U.S. Cl. CPC A61K 8/046; A61K 8/8129; A61Q 19/10; A61Q 19/007; A61K 2800/28; A61K 2800/70; A61K 2800/412; A61K 2800/805

(57) ABSTRACT

Embodiments of the disclosure relate to dissolvable solid foam compositions having a soluble polymer and a surfactant. The compositions may be biodegradable and a closed cell solid foam. Also described are kits, methods of preparation and methods of use of such dissolvable solid foam compositions.

### TECHNICAL FIELD

Embodiments of the present disclosure relate to dissolvable solid foam compositions, for example, biodegradable dry foam soaps, shaving creams, moisturizers, sunscreens, etc. used for personal care and cleansing. Also described are methods of preparation and kits thereof.

### BACKGROUND

Many personal care products and cleansers are in the form of liquids. Conventional liquid products often contain compounds that are non-biodegradable, which can increase the downstream cost of water treatment to remove such compounds. Liquid products also can be difficult to package, store and transport without leaking. The bottles used to store liquid products contribute to their cost and increase waste as the bottles frequently end up in landfills. The aqueous fluids and solvents used in personal care products increase the weight and size of the products and result in extra packaging, shipping and storage costs.

There is a need for dissolvable solid foam compositions that are biodegradable, can reduce packaging as well as shipping and storage costs, and can reduce solid and liquid waste as compared to liquid formulations.

### SUMMARY

According to one or more embodiments, disclosed herein is dissolvable solid foam composition, comprising: a soluble polymer; and a surfactant, wherein the composition is a closed cell foam.

In some embodiments, disclosed herein is a biodegradable solid foam composition, comprising: a soluble polymer comprising polyvinyl alcohol; a surfactant comprising an ethoxylate; and an exfoliant powder, wherein the solid foam composition biodegrades in less than about 1 year.

In at least one embodiment, disclosed herein is a personal care product, comprising: a dissolvable solid foam composition according to one or more embodiments described herein; and a sachet overlaying the dissolvable solid foam composition, wherein the sachet comprises a soluble polymer.

According to one or more embodiments, disclosed herein is kit, comprising: a plurality of pieces of a dissolvable solid foam composition according to one or more embodiments described herein; a plurality of sachets, each sachet comprising a soluble polymer and configured to overlay at least one of the plurality of pieces; and instructions to: contact at least one of the plurality of pieces and a corresponding sachet with water to form a cleansing composition within about 5 sec to about 60 sec; apply the cleansing composition to skin or hair for at least 30 sec; and rinse the cleansing composition with water.

### BRIEF DESCRIPTION OF THE DRAWINGS

The present disclosure is illustrated by way of example, and not by way of limitation, in the figures of the accompanying drawings in which like references indicate similar elements. It should be noted that different references to “an” or “one” embodiment in this disclosure are not necessarily to the same embodiment, and such references mean at least one.

FIG. 1A shows a top view of a tray baked sample of a dissolvable solid foam composition according to one or more embodiments.

FIG. 1B shows a bottom view of a tray baked sample of a dissolvable solid foam composition according to one or more embodiments.

FIG. 1C shows a side view of a tray baked sample of a dissolvable solid foam composition according to one or more embodiments.

FIG. 2A shows an optical microscopy image of a dissolvable solid foam composition containing various solid minerals and exfoliants according to one or more embodiments.

FIG. 2B shows an optical microscopy image of a dissolvable solid foam composition containing various solid minerals and exfoliants according to one or more embodiments.

FIG. 2C shows an optical microscopy image of a dissolvable solid foam composition containing various solid minerals and exfoliants according to one or more embodiments.

FIG. 3A shows an optical microscopy image of a dissolvable solid foam composition having a low density according to one or more embodiments.

FIG. 3B shows an optical microscopy image of a dissolvable solid foam composition having a low density according to one or more embodiments.

FIG. 4A shows an optical microscopy image of a dissolvable solid foam composition having a high density according to one or more embodiments.

FIG. 4B shows an optical microscopy image of a dissolvable solid foam composition having a high density according to one or more embodiments.

FIG. 4C shows an optical microscopy image of a dissolvable solid foam composition having a high density according to one or more embodiments.

### DETAILED DESCRIPTION

Disclosed herein are embodiments of a dissolvable solid foam composition and methods of preparation and use thereof. The composition may be rapidly dissolvable, for example, able to dissolve in less than about 30 seconds, less than about 20 seconds, less than about 10 seconds, less than about 5 seconds, less than about 1 second, or within about 1 second to about 120 seconds, about 5 seconds to about 60 seconds, or any individual value or sub-range within these ranges, when contacted by one or more of water, an aqueous liquid, a solvent and/or an acid. The composition may contain biodegradable, bioresorbable (e.g., absorbed back into the environment), naturally occurring and/or ecologically friendly components that reduce environmental and ecological issues associated with conventional consumer cleansing products. The term “biodegradable” as used herein may refer to the length of time it takes for a chemical compound to fully degrade to its basic components (e.g., natural minerals, carbon dioxide, water) in water, that is, a chemical compound may be biodegradable if it takes less than about one (1) year, or less than about 9 months, or less than about 6 months, or less than about 3 months, or about one hour to about three months to completely degrade in water, or any individual time or sub-range within these ranges. Examples of biodegradable compounds include, but are not limited to, aconityls, acetals, amides, carboxylic anhydrides, esters, imines, hydrazones, maleamic acid amides, ortho esters, phosphamides, phosphoesters, phosphosilyl esters, silyl esters, sulfonic esters, aromatic carbamates, and combinations thereof.

The term “readily biodegradable” us used herein may refer to an article or composition that is tested in accordance with an OECD 301B biodegradation test and achieves a classification of Ready Biodegradability. The pass levels for ready biodegradability can be 70% removal of discrete organic chemicals (DOC) and 60% of theoretical oxygen demand (ThOD) or theoretical carbon dioxide demand (ThCO2) production for respirometric methods. These pass values can be reached in a 10-d window within the 28-d period of the test. In some embodiments, an article or composition is readily biodegradable if it achieves greater than about 50%, greater than about 60%, greater than about 70%, greater than about 80%, greater than about 90% biodegradation, or any percent or sub-range within these ranges, within a period of less than about 1 year, less than about 9 months, less than about 6 months, less than about 3 months, less than about 2 months, less than about 1 month, less than about 28 days, or less than about 10 days.

The term “ecologically friendly,” which herein may be used interchangeably with the terms “environmentally friendly” or “environmentally preferable,” refers to products or services that have a lesser or reduced effect on human health and the environment when compared with competing products or services that serve the same purpose.

Dissolvable solid foam compositions according to embodiments herein may provide an invigorating experience to a user as a result of one or more of foaming agents, essential oils, and/or other compounds incorporated therein. In at least one embodiment, the solid foam compositions may be small, having a volume of about 1 cm³ to about 100 cm³, about 5 cm³ to about 75 cm³, or any individual volume or sub-range within these ranges, about 10 cm³ to about 50 cm³, or about 20 cm³, about 30 cm³, about 40.5 cm³, about 50 cm³. In some embodiments, the solid foam composition may have a mass of about 0.5 g to about 10 g, or about 1.0 g to about 5.0 g, or any individual mass or sub-range therein. As discussed above, the dissolvable solid foam compositions may dissolve and become a liquid quickly when contacted with water or other solvent, for example, in a shower, tap, or other source. According to one or more embodiments, the dissolvable solid foam compositions can be formulated as a shampoo, body cleanser, face cleanser, hand cleanser, shaving cream, moisturizer, sun block, conditioner, and/or other personal care product.

According to one or more embodiments, dissolvable solid foam compositions may include a foam cleanser formulation including a soluble polymer (e.g., a water-soluble polymer) additive. During processing of the dissolvable solid foam composition, the foam cleanser formulation produces bubbles and enables the composition to set as a solid unit having a plurality of air pockets (i.e., as a solid foam structure). The air pockets in the dissolvable solid foam compositions may have a generally circular or ovular cross-section with a mean diameter of about 0.1 μm to about 1,000 μm, about 1 μm to about 500 μm or any individual value or sub-range within these ranges. The air pockets provide the dissolvable solid foam composition with a low overall density (and weight) and a large surface area that enables rapid dissolution. The air pockets may be present in the dissolvable solid foam composition in an amount of about 25% to about 95% by volume of the total volume of the dissolvable solid foam composition, or any individual value or sub-range within these range. The large surface area and open air volume of the composition allows water to quickly enter and dissolve the solid foam composition.

According to at least one embodiment, the dissolvable solid foam compositions may be comprised of a single piece of solid foam as a unit or of about 2 to about 10 individual sheets of solid foam composition, or any individual value or sub-range therein. The sheets may be attached and/or adhered to each other to form a stack of layers. In one or more embodiments, the sheets can be adhered together using a water-soluble spray adhesive, calendaring or solvent misting to fuse the layers together. A single layer can be a flat sheet having an area of about 10 cm² to about 40 cm², or any individual value or sub-range therein. One or more sheet may have a weight of about 0.5 gram to about 10 grams, or any individual value or sub-range therein. Each sheet may have a thickness of about 0.1 mm to about 5 mm, or any individual value or sub-range therein, and can be in any suitable shape (e.g., rectangular, square, triangular, circular, diamond, and so on). The density of each sheet can be about 0.005 g/cc to about 0.75 g/cc, or any individual value or sub-range therein.

In one or more embodiments, the dissolvable solid foam compositions may include pinholes or piercings, in addition to the plurality of air pockets, throughout the composition. The pinholes and layers allow pathways for the aqueous liquid to enter the solid foam structure to increase the dissolution speed. The pinholes or piercings may be randomly arranged throughout the three-dimensional solid foam structure or may be arranged in a regular pattern, for example, evenly spaced apart in rows, columns, etc. The solid foam structure may have additional pinholes or piercings throughout the material. The number, frequency and/or spacing of the pinholes can increase water intercalation and dissolution speed of the solid foam structure as compared to a solid foam composition without such pinholes.

According to one or more embodiments, the solid foam composition may have a closed cell structure. The term “closed cell” as used herein may refer to cells or air pockets within the solid foam structure that are entirely enclosed by their walls and do not interconnect with other cells. A closed cell bubble may be completely encased by solid. A closed cell solid foam according to embodiments herein may have greater than 50%, greater than about 60%, greater than about 70%, greater than about 75%, greater than about 80% or greater than about 90% of non-interconnecting cells, or any individual percentage or sub-range within these ranges, as measured by a standardized method for measuring rigid cellular plastics to determine the volume percentage of open cells and of dosed cells (e.g., ISO 4590:2016) or a standard test method for open cell content of rigid cellular plastics (e.g., ASTM D6226-15).

As discussed above, dissolvable solid foam compositions according to one or more embodiments herein, include a foam cleanser formulation. Components of the dissolvable solid foam compositions, including the foam cleanser formulation, may be natural, naturally derived, biodegradable, vegan, gluten free, plant-based and/or mineral-based.

In one or more embodiments, the foam cleanser formulation includes one or more of a surfactant, a fragrance, a solid, a moisturizer and/or a vitamin. Suitable surfactants for use in the foam cleanser formulation include, but are not limited to, a saponin (e.g., one or more of a triterpene glycoside), an alcohol ethoxylate, an oil ethoxylate, lauramine oxide, a lauryl alcohol ethoxylate and/or combinations of any two or more of the foregoing. Suitable alcohol ethoxylates include, but are not limited to, fatty alcohol ethoxylates, fatty acid ethoxylates, polyethoxylated oleyl alcohols (e.g. BRIJ® 92 and SIMUSOL® 92, ethoxylated castor oil, ethyoxylated soybean oil, propoxylated castor oil, propoxylated soybean oil, ethoxylated glyceryl monostearate, C₈-C₃₀ alcohol ethoxylate, octyl alcohol ethoxylate, lauryl alcohol ethoxylate, myristyl alcohol ethoxylate, cetyl alcohol ethoxylate, stearyl alcohol ethoxylate, cetearyl alcohol alcohol ethoxylate, sterol ethoxylates, oleyl alcohol ethoxylate, alkyl phenolic alkoxy, octylphenol ethoxylate, polyoxyethylene-polyoxypropylene block copolymer, polyoxyethylene glycol and/or combinations of any two or more of the foregoing. In one or more embodiment, the dissolvable solid foam composition may contain one or more surfactant in an amount of about 1% by weight to about 99% by weight based on the total weight of the dissolvable solid foam composition, or less than 23% by weight of the total weight of the composition, or greater than 75% by weight of the total weight of the composition or any individual value or sub-range therein.

In one or more embodiments, the dissolvable solid foam composition may be in the form of a shaving cream. Such compositions can include, but are not limited to, citric acid, hair keratin amino acids, stearic acids, boric acid, fumaric acid, SD Alcohol 40, cetyl alcohol, stearyl alcohol, calcium chloride, sodium chloride, camphor, hydroxyethylcellulose, calcium hydroxide, ammonium hydroxide, triethanolamine, gelatin, stearamidopropyl dimethylamine, methylchloro-isothiazolone, methylisothiazolinone, cyclopentasiloxane, glycerin, dimeticonol, linseed oil, soybean oil, eucalyptus oil, propylene glycol, fragrance, menthol, guar gum, DMDM hydantoin, sodium calcium alginate, panthenol, oleyl sarcosine, methylparaben, stearyl octyldimonium methosulfate, behenamidopropyl ethyldimonium ethosulfate, TEA-dodecylbenzenesulfonate, calcium sulfate and disodium EDTA; and/or an emulsifier.

Suitable fragrances include, but are not limited to, a perfume, an essential oil, an extract, an absolute, a resinoid, a concentrate, eucalyptus globulus leaf oil (i.e., eucalyptus), prunus amygdalus dulcis (e.g., sweet almond) oil, lavender oil, rosemary oil, vanilla oil, tea tree oil, peppermint oil, spearmint oil, sweet orange oil, lemon oil and/or combinations thereof. In one or more embodiment, the dissolvable solid foam composition may contain one or more fragrance in an amount of less than about 10 wt %, less than about 5 wt %, less than about 2 wt %, less than about 1 wt %, or about 0.001 wt % to about 1 wt % based on the total weight of the dissolvable solid foam composition, or any individual value or sub-range therein.

Suitable solids for use in the foam cleanser formulation include, but are not limited to, an exfoliant, rough grains, seed meal, corn meal, coffee grounds, sand, activated charcoal and/or combinations thereof. The solids may be in the form of particles, granules, pellets, extrudates, beads and/or combinations thereof. In some embodiments, the solids may be in the form of a exfoliant powder having a particle, granule, pellet, extrudate and/or bead size distribution (collectively referred to as a particle size distribution) of less than about 500 μm, less than about 250 μm, or with a mean size (or collectively referred to as a mean particle size) of about 1 μm to about 500 μm. In one or more embodiment, the dissolvable solid foam composition may contain one or more solids in an amount of less than about 15 wt %, less than about 10 wt %, less than about 5 wt %, less than about 2 wt %, less than about 1 wt %, or about 0.001 wt % to about 1 wt % based on the total weight of the dissolvable solid foam composition, or any individual value or sub-range therein.

Suitable moisturizers and/or vitamins for use in the foam cleanser formulation include, but are not limited to, sodium hyaluronate, aloe, vitamin E oil (e.g., one or more tocopherol and one or more tocotrienol), shea oil, olive oil, jojoba oil, sesame oil, rosehip seed oil, apricot kernel oil, coconut oil, sweet almond oil, sandalwood oil, jasmine oil, juniper oil, vitamin A (e.g., one or more of retinol, retinal and/or retinyl esters), vitamin B3 (i.e., niacin), L-ascorbic acid (vitamin C), zinc and/or combinations thereof. In one or more embodiment, the dissolvable solid foam composition may contain one or more moisturizer in an amount of about 1 wt % to about 50 wt % based on the total weight of the dissolvable solid foam composition, or any individual value or sub-range therein. In one or more embodiment, the dissolvable solid foam composition may contain one or more vitamin in an amount of about 1 mcg to about 1000 mcg, or any individual value or sub-range therein.

The foam cleanser formulation in solid foam structure may further include, be enclosed by, coated with and/or overlaid with a soluble polymer. In one or more embodiments, the solid foam structure may be enclosed in a sachet formed of the soluble polymer. In some embodiments, the soluble polymer may be combined with the other components of the foam cleanser formulation and may function as a structural component that helps set the solid foam during processing yet is also rapidly dissolvable when exposed to water, an aqueous fluid, a solvent and/or a acid.

Suitable soluble polymers for use in one or more embodiments of dissolvable solid foam compositions include, but are not limited to, polyvinyl pyrrolidone (“PVP”), polyvinyl alcohol (“PVA”), cellulose acetate (e.g., water-soluble cellulose acetate), modified wood pulp, modified paper pulp and/or combinations thereof. In one or more embodiment, the dissolvable solid foam composition may contain the soluble polymer in an amount of about 1% by weight to about 99%, or about 30% by weight to about 70% by weight based on the total weight of the dissolvable solid foam composition, or less than 30% by weight of the total weight of the composition, or greater than 70% by weight of the total weight of the composition or any individual value or sub-range therein.

A variety of polymer grades can be used for one or more component of the soluble polymer in the foam cleanser formulation. Table 1 shows several suitable PVA grades and their anticipated effect on dissolution of the dissolvable solid foam composition. Similar variations in polymer grades for PVP or cellulose acetate can be used to tune properties of the foam product.

TABLE 1
Suitable Grades of Polyvinyl Alcohols for the Soluble Polymer

| PVA Product Name | Hydrolysis (%) | Viscosity (cP)* | Dissolution Properties |
|---|---|---|---|
| PVA 1788L | 86 (measured) | 23 (measured) | Standard PVA |
| (088-20) | 88 (specified) | 20-26 (specified) | |
| PVA 2088 | 88 | 33 | Higher molecular weight than |
| (088-35) | | | standard PVA but same |
| | | | hydrolysis; |
| | | | likely to dissolve more slowly |
| | | | than standard PVA, but may |
| | | | have a “thicker” feel |
| PVA 2488 | 88 | 50 | Highest bulk-available |
| (088-50) | | | molecular weight, but same |
| | | | hydrolysis as standard PVA; |
| | | | will likely dissolve slower than |
| | | | current sample and have a |
| | | | “thicker” feel |
| PVA 1799 | 99 | 25 | Similar molecular weight as |
| (088-20) | | | standard PVA, but a higher |
| | | | hydrolysis; |
| | | | will likely dissolve much |
| | | | slower than standard PVA, but |
| | | | once dissolved may have a |
| | | | similar feel |
| PVA 1792 | 92 | 20-30 | Similar molecular weight as |
| (092-20) | | | standard PVA, but a modestly |
| | | | higher hydrolysis; |
| | | | will dissolve a bit slower than |
| | | | standard PVA, but may have a |
| | | | similar feel |
| PVA 0588 | 88 | 4-6 | Will likely dissolve faster than |
| (088-05) | | | standard PVA, but may have a |
| | | | “thinner” feel/more glide. |

*Viscosity was measured of a 4 wt % PVA solution in water at room temperature

Further disclosed herein are kits including the dissolvable solid foam compositions according to embodiments herein. As discussed above, in one or more embodiments, the foam cleanser formulation in solid foam structure may be packaged in a water-soluble biodegradable sachet to assist with handling. Formulating the packaging from a water-soluble material helps maintain the functionality of the composition including the rapid dissolution properties. Suitable water-soluble materials for use as a packaging material are the same as the soluble polymers as described herein. The sachet material will generally dissolve in water in less than about 90 seconds, or less than about 60 seconds, or less than about 30 seconds, or less than about 15 seconds, or less than about 1 second, or in about 0.1 second to about 90 seconds. Kits as disclosed herein my include one or more dissolvable solid foam compositions each individually packaged in a water-soluble biodegradable sachet.

According to at least one embodiment, a composition may be in the form of a shaving formulation (e.g., a dry foam shaving composition). Such shaving formulations can include any one or more of the components described above. In some embodiment, shaving formulations can include, but are not limited to, solvent(s) in an amount of about 1 wt % to about 20 wt %, film former(s) in an amount of about 20 wt % to about 35 wt %, anionic surfactant(s) in an amount of about 1 wt % to about 50 wt %, skin protectant(s) in an amount of about 1 wt % to about 20 wt %, humectant(s) in an amount of about 0.5 wt % to about 10 wt %, preservative(s) in an amount of about 0.5 wt % to about 10 wt %, bulking agent(s) in an amount of about 0.01 wt % to about 1.5 wt %, skin conditioning agent(s) in an amount of about 0.1 wt % to about 5 wt %, or combinations thereof, each component in any individual amount or sub-range within the aforementioned ranges. In some embodiments, the shaving formulation can include, but is not limited to, water, polyvinyl alcohol, coco glucoside, Zea mays (corn) starch, sodium methyl cocoyl isethionate, glycerin, sodium chloride, potassium cocoyl glycinate, calcium sulfate, lavandul angustifolia (lavender) flower extract, camellia japonica flower extract, Lonicera japonica (honeysuckle) flower extract, Triticum vulgare (wheat) germ extract, Brassica oleracea italic (broccoli) sprout extract, allantoin or combinations thereof.

Further disclosed herein are methods of manufacturing dissolvable solid foam compositions using a tray baking method. The tray baking method may include mixing a soluble polymer, a surfactant and one or more other additives (e.g., a fragrance, vitamin, solids, moisturizers, etc.) in a solvent (e.g., water) to form a solution. A weight ratio of the soluble polymer to the surfactant may be about 1:50 to about 50:1, about 1:40 to about 40:1, about 1:25 to about 25:1, about 1:10 to about 10:1, about 1:1 to about 50:1, about 50:1 to about 1:1, or any individual weight ratio or sub-range within these ranges. The solution may be mixed thoroughly, for example, using a mechanical stirrer, magnetic stirrer, agitation, sparging, laminar flow and/or combinations thereof. Suitable mixing methods can include aeration, turbulent mixing or shear mixing to form air bubbles in the solution.

The method can further include transferring (e.g., pouring) the solution into one or more baking trays or molds and baking the solution and trays in an oven to evaporate water and/or moisture from the solution. Water evaporation and/or boiling can form additional air pockets in the solution in addition to air bubbles formed during mixing. Baking the solution may be performed at a temperature of about 90° C. to about 180° C. for about 15 minutes to about 3 hours depending on the temperature and desired thickness of the product. After baking, the solution becomes a solid foam structure suitable as a dissolvable solid foam composition according to embodiments. The resulting solid foam structure may be a cuboid or other three-dimensional structure resulting from the shape of the baking tray. The method may further include cutting, breaking and/or otherwise dividing the solid foam structure into a plurality of pieces having a desired size, volume, and/or weight.

In one or more embodiments, a roll sheet baking method may be used to prepare dissolvable solid foam compositions according to embodiments herein. The roll sheet baking method may include combining one or more of a soluble polymer, a surfactant, and/or other additives (e.g., a fragrance, vitamin, solids, moisturizers, etc.) in solvent (e.g., water) to form a solution. The solution may be mixed thoroughly, for example, using a mechanical stirrer, magnetic stirrer, agitation, sparging, laminar flow and/or combinations thereof. Suitable mixing methods can include aeration, turbulent mixing or shear mixing to form air bubbles in the solution.

The mixed solution can be poured in a continuous or semi-continuous manner onto a heated roll of a roll heating system or heated belt. The heated roll may be at a temperature of about 120° C. to about 200° C. As the mixed solution dries and cures on the heated roll, it may be in contact with the roll for a period of about 15 minutes to about 3 hours depending on the temperature, desired thickness and speed of the roll. As the mixed solution is heated by the roll, water and other moisture within the solution evaporates and dries the solution to form the solid foam structure. Evaporation of water and/or boiling can add additional air pockets in addition those generated during mixing.

The resulting solid foam structure may be a cuboid or other three-dimensional structure resulting from the shape of the baking tray. The method may further include cutting, breaking and/or otherwise dividing the solid foam structure into a plurality of pieces having a desired size, volume, and/or weight.

Further disclosed herein are methods of manufacturing dissolvable solid foam compositions using a melt cast method. The method can include mixing one or more of a soluble polymer, a surfactant and/or other additives (e.g., a fragrance, vitamin, solids, moisturizers, etc.) in a solvent (e.g., water, if used) to form a solution of about 0 wt % to about 50 wt % solvent, for example, so that at room temperature the solution remains a solid or thick paste. The method further includes heating the solution at a temperature of about 90° C. to about 200° C. and stirring the solution until it melts. The method can include introducing air bubbles into the melted solution, for example, by aeration, turbulent mixing and/or shear mixing, while staying above the melting temperature of the solution. The melted solution containing air bubbles may be poured into one or more trays or molds. The trays or molds may be maintained at a temperature below the melting temperature of the solution until the melted solution cures and forms the solid foam structure.

The resulting solid foam structure may be a cuboid or other three-dimensional structure resulting from the shape of the baking tray. The method may further include cutting, breaking and/or otherwise dividing the solid foam structure into a plurality of pieces having a desired size, volume, and/or weight.

Further disclosed herein are methods of manufacturing dissolvable solid foam compositions using a freeze cast method. The roll sheet baking method may include combining one or more of a soluble polymer, a surfactant, and/or other additives (e.g., a fragrance, vitamin, solids, moisturizers, etc.) in solvent (e.g., water) to form a solution. The solution may be mixed thoroughly, for example, using a mechanical stirrer, magnetic stirrer, agitation, sparging, laminar flow and/or combinations thereof. Suitable mixing methods can include aeration, turbulent mixing or shear mixing to form air bubbles in the solution. The mixed solution containing air bubbles may be poured into one or more trays or molds. The trays or molds may be freeze dried (i.e., frozen and placed under vacuum) to evaporate and remove water and other moisture.

The resulting solid foam structure may be a cuboid or other three-dimensional structure resulting from the shape of the baking tray. The method may further include cutting, breaking and/or otherwise dividing the solid foam structure into a plurality of pieces having a desired size, volume, and/or weight.

Further disclosed herein are methods of manufacturing dissolvable solid foam compositions using a lost melt cast method. The method may including mixing one or more of a soluble polymer, a sacrificial polymer, a surfactant, and/or other additives (e.g., a fragrance, vitamin, solids, moisturizers, etc.) with or without a solvent (e.g., water or other fluid) to form a solution. Suitable sacrificial polymers include, but are not limited to, polyethylene glycol having a molecular weight of about 1,000 Da to about 16,000 Da, polyethylene oxide having a molecular weight of about 16,000 Da to about 10,000,000 Da, polyvinyl alcohol (e.g., having a hydrolysis content of 0-99% and a molecular weight of 5,000-200,000 Da), polyacrylic acid or a sodium salt thereof (e.g., having a molecular weight of 5,000-3,000,000 Da), polyacrylamide (e.g., having a molecular weight of 40,000-150,000 Da), polypeptides or salts thereof, such as polyaspartic acid, polyglutamic acid, polylysine HCl or polylysine HBr (e.g., having a molecular weight of 2,000-150,000 Da), gelatin, laminin or albumin, water soluble cellulose derivatives, such as ethylcellulose, methylcellulose, sodium carboxymethylcellulose, ethyl hydroxyethyl cellulose, hydroxypropylcellulose, or hydroxyethylcellulose (e.g., with modification contents of 40-99% and molecular weights of 1,000-1,000,000 Da), polysaccharides, such as sodium alginate, hyaluronic acid or a sodium salt thereof, pectin, guar gun, xantham gum, chitosan, agar, water-soluble modified starch, such as dextrin, maltodextrin, starch acetate, carboxy/methoxy ethylated or methylated starch or cationic starch, and/or any combination of two or more of the foregoing.

In some embodiments, the sacrificial polymer includes, but is not limited to, polyvinyl alcohol having a hydrolysis content of 0-99% and a molecular weight of 5,000-200,000 Da, polyacrylic acid, or sodium salt thereof, having a molecular weight of 5,000-3,000,000 Da, polyacrylamide having a molecular weight of 40,000-150,000 Da; polypeptides, or salts thereof, such as polyaspartic acid, polyglutamic acid, polylysine HCl or polylysine HBr, having a molecular weight of 2,000-150,000 Da, gelatin, laminin or albumin; water soluble cellulose derivatives such as ethylcellulose, methylcellulose, sodium carboxymethylcellulose, ethyl hydroxyethyl cellulose, hydroxypropylcellulose, or hydroxyethylcellulose, with modification contents of 40-99% and molecular weights of 1,000-1,000,000 Da; polysaccharides such as sodium alginate, hyaluronic acid, or sodium salt thereof, pectin, guar gun, xantham hum, chitosan, agar; or water-soluble modified starch such as dextrin, maltodextrin, starch acetate, carboxy/methoxy ethylated or methylated starch, or cationic starch.

According to one or more embodiments, the sacrificial polymer is not miscible at room temperature with the other components in the solution. The solution may contain about 10 wt % to about 40 wt %, about 15 wt % to about 30 wt %, or any individual weight percent or sub-range within these ranges, of the sacrificial polymer. The method further includes heating the solution at a temperature of about 90° C. to about 200° C. and stirring the solution until it forms a melts (e.g., a uniform melt). The method can include introducing air bubbles into the melted solution, for example, by aeration, turbulent mixing and/or shear mixing, while staying above the melting temperature of the solution. The melted solution containing air bubbles may be poured into one or more trays or molds. The trays or molds may be maintained at a temperature below the melting temperature of the solution until the melted solution dries and cures.

The resulting solid foam structure may be a cuboid or other three-dimensional structure resulting from the shape of the baking tray. The method may further include cutting, breaking and/or otherwise dividing the solid foam structure into a plurality of pieces having a desired size, volume, and/or weight.

The method can further include removing the sacrificial polymer by contacting the dried and cured solution with a solvent that dissolves the sacrificial polymer, but does not dissolve the soluble polymer or components within the solution. Alternatively, the dried and cured solution may be heated at a temperature of about 40° C. to about 80° C. to melt the sacrificial polymer, but below the melting temperature for the other components in the solution, which remain solid. The removal of the sacrificial polymer leaves behind a porous network foam forming the dissolvable solid foam composition.

As discussed above, the dissolvable solid foam composition may be formed from a plurality of sheets or layers of the solid foam structure. The individual sheets may be attached using one or more of a water soluble spray adhesive, calendaring and/or solvent-misting, for example, to fuse the layers together. The resulting stack of connected sheets forms the dissolvable solid foam composition in suitable form for packaging, distribution and use.

As further discussed above, the once dried, cured and/or formed into a stack, the solid foam structure may be pierced and/or punctured, for example, using a needle to form pin holes in the material. The pin holes in addition to the air pockets resulting from the preparation process may further increase the dissolution rate of the closed cell structure as compared to a pin hole free composition. The pin holes may have a diameter of less than about 2 mm, less than about 1 mm, less than about 0.5 mm, less than about 0.1 mm, less than about 500 μm, less than about 100 μm, less than about 10 μm, or about 1 μm to about 2 mm, or any individual diameter or sub-range within this range.

The solid foam structures resulting from any of the above-described methods may be individually packaged in sachets formed of a soluble polymer. In some embodiments, the sachets are pre-formed “bags” sized and configured to receive a single solid foam structure. In some embodiments, the soluble polymer may be applied to the individual solid foam structures in liquid form and allowed to form, dry and seal about the structures.

Dissolvable solid foam compositions according to embodiments herein may be used for cleansing and otherwise treating mammalian keratinous tissue such as hair and/or skin, and can rapidly dissolve to provide lathering. A method for cleansing skin or hair may comprise the steps of: a) holding a dissolvable solid foam composition in a hand, b) wetting the dissolvable solid foam composition with water and rubbing the solid foam structure between hands, fingers or on the skin to dissolve, c) applying the dissolved material to skin or hair to cleanse and/or treat, and d) rinsing the dissolved composition from the hair or skin using water. These steps can be repeated as desired to achieve sufficient cleansing and/or or treatment benefits.

The amount of the composition applied, the frequency of its application and the length of use may vary depending on the desired effect, the concentration of ingredients in a composition and the level of regulation desired. For example, when the composition is applied for whole body or hair treatment, effective amounts are about 0.5 g to about 10 g, or any individual amount or sub-range therein.

### Illustrative Examples

The following examples are set forth to assist in understanding the disclosure and should not be construed as specifically limiting the disclosure described and claimed herein. Such variations of the disclosure, including the substitution of all equivalents now known or later developed, which would be within the purview of those skilled in the art, and changes in formulation or minor changes in experimental design, are to be considered to fall within the scope of the disclosure incorporated herein.

### Example 1—A Dissolvable Solid Foam Composition

A dissolvable solid foam composition according to one or more embodiments described herein was prepared. The composition was prepared by combining the ingredients as shown in Table 2. The ingredients were combined with water at 30.7 wt % solids loading and whipped with an immersion blender. Subsequently, 6.12 g of pre-baking mixture were poured into 2 in×2 in silicone molds and baked at a temperature of 150° C. for 2 hours. The average mass of the samples after baking was 1.88 g. The ingredients in the final produce are shown in Table 3.

TABLE 2
Ingredients (pre-baking)

| Component | Weight % |
|---|---|
| Disodium Laureth Sulfosuccinate | 1.0 |
| Sodium Lauryl Sulfoacetate | 0.5 |
| Polyvinyl Alcohol (Eastchem 1788L) | 12.8 |
| Sodium Cocoyl Isethionate | 8 |
| Lauryl Glucoside | 3.2 |
| Zea Mays (Corn) Starch | 4.3 |
| Sodium Chloride | 0.9 |
| Water | 69.3 |

TABLE 3
Ingredients (final product)

| Component | Weight % |
|---|---|
| Disodium Laureth Sulfosuccinate | 3.3 |
| Sodium Lauryl Sulfoacetate | 1.7 |
| Polyvinyl Alcohol (Eastchem 1788L) | 41.8 |
| Sodium Cocoyl Isethionate | 26.0 |
| Lauryl Glucoside | 10.4 |
| Zea Mays (Corn) Starch | 13.9 |
| Sodium Chloride | 2.8 |

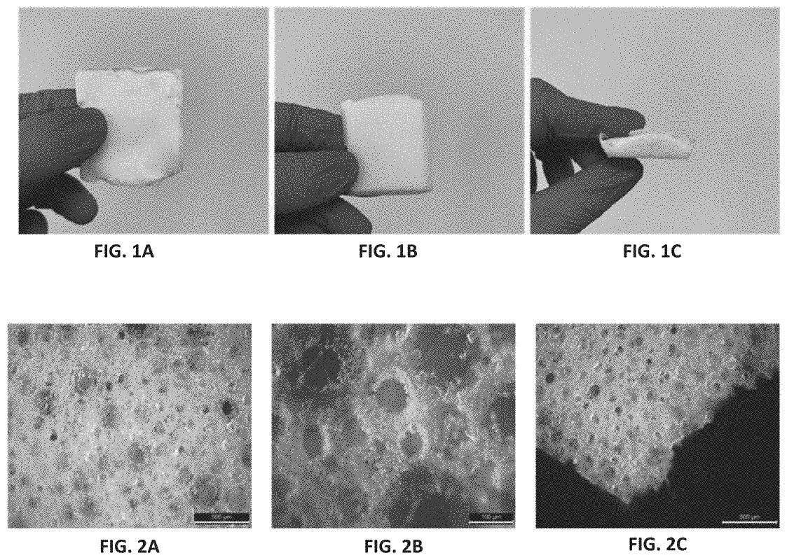

FIGS. 1A-1C show images of the resulting dissolvable solid foam compositions. As shown in the images, the resulting solid foam structure had a foam-like appearance and was small and light enough to be easily held by two digits of a human hand. The dissolvable solid foam composition may be easily rubbed against one's skin as a cleanser when bathing or washing.

### Example 2—A Dissolvable Solid Foam Composition

A dissolvable solid foam composition was prepared according to the method as described in Example 1. The ingredients of the solid foam composition include solid minerals, exfoliants and water. FIGS. 2A-2C show optical microscopy images of the resulting dissolvable solid foam compositions. As shown in the images, the resulting solid foam structure was closed cell. The air pockets formed in the solid foam structure were less than about 250 μm.

### Example 3—A Dissolvable Solid Foam Composition (Low Density)

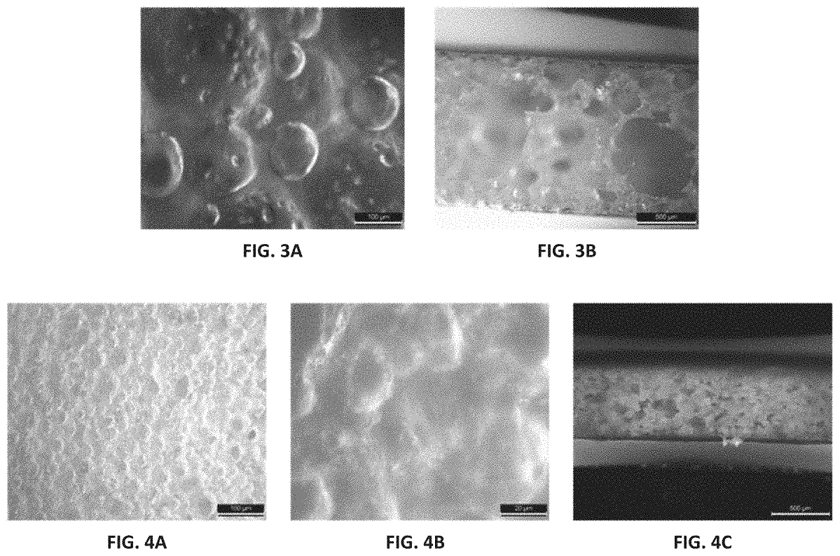

A dissolvable solid foam composition having a low density of about 0.01 g/cc to about 0.08 g/cc was prepared according to the method as described in Example 1. FIGS. 3A-3B show optical microscopy images of the resulting low density dissolvable solid foam compositions. As shown in the images, the resulting solid foam structure was a closed cell foam. The air pockets formed in the solid foam structure were less than about 750 μm.

### Example 4—A Dissolvable Solid Foam Composition (High Density)

A high density dissolvable solid foam composition was prepared according to the method described in Example 1. FIGS. 4A-4C show optical microscopy images of the resulting dissolvable solid foam compositions. As shown in the images, the resulting solid foam structure was closed cell. The air pockets formed in the solid foam structure were less than about 50 μm.

### Example 5—A Dissolvable Solid Foam Shaving Composition (Prophetic)

A shaving foam composition according to various embodiments described herein may be prepared by one or more of the methods according to embodiments herein. The shaving foam composition has the ingredients as set forth in Table 4.

TABLE 4
Shaving Foam Composition

| Ingredient | Wt (%) | Function |
|---|---|---|
| Water | 1.0000-20.000 | Solvent |
| Polyvinyl Alcohol | 10.000-35.000 | Film Former |
| Coco Glucoside | 10.000-35.000 | Anionic Surfactants |
| Zea Mays (Corn) Starch | 1.0000-20.000 | Skin Protectant |
| Sodium Methyl Cocoyl Taurate | 1.0000-15.000 | Anionic Surfactants |
| Sodium Cocoyl Isethionate | 1.0000-15.000 | Anionic Surfactants |
| Glycerin | 1.0000-10.000 | Humectant |
| Sodium Chloride | 1.0000-10.000 | Preserved Agent |
| Potassium Cocoyl Glycinate | 1.0000-10.000 | Anionic Surfactants |
| Calcium Sulfate | 0.10000-5.0000 | Bulking Agent |
| Lavandula Angustifolia | 0.01000-5.0000 | Skin Conditioning |
| (Lavender) Flower Extract | | Agent |
| Camellia Japonica Flower | 0.01000-5.0000 | Skin Conditioning |
| Extract | | Agent |
| Lonicera Japonica | 0.01000-5.0000 | Skin Conditioning |
| (Honeysuckle) Flower Extract | | Agent |
| Triticum Vulgare (Wheat) Germ | 0.01000-5.0000 | Skin Conditioning |
| Extract | | Agent |
| Brassica Oleracea Italica | 0.01000-5.0000 | Skin Conditioning |
| (Broccoli) Sprout Extract | | Agent |
| Allantoin | 0.01000-5.0000 | Skin Conditioning |
| | | Agent |

The preceding description sets forth numerous specific details such as examples of specific systems, components, methods, and so forth, in order to provide a good understanding of several embodiments of the present disclosure. It will be apparent to one skilled in the art, however, that at least some embodiments of the present disclosure may be practiced without these specific details. In other instances, well-known components or methods are not described in detail or are presented in simple block diagram format in order to avoid unnecessarily obscuring the present disclosure. Thus, the specific details set forth are merely exemplary. Particular implementations may vary from these exemplary details and still be contemplated to be within the scope of the present disclosure.

As used herein, the singular forms “a,” “an,” and “the” include plural references unless the context clearly indicates otherwise. Thus, for example, reference to “a precursor” includes a single precursor as well as a mixture of two or more precursors; and reference to a “reactant” includes a single reactant as well as a mixture of two or more reactants, and the like.

Reference throughout this specification to “one embodiment” or “an embodiment” means that a particular feature, structure, or characteristic described in connection with the embodiment is included in at least one embodiment. Thus, the appearances of the phrase “in one embodiment” or “in an embodiment” in various places throughout this specification are not necessarily all referring to the same embodiment. In addition, the term “or” is intended to mean an inclusive “or” rather than an exclusive “or.” When the term “about” or “approximately” is used herein, this is intended to mean that the nominal value presented is precise within ±10%, such that “about 10” would include from 9 to 11.

The term “at least about” in connection with a measured quantity refers to the normal variations in the measured quantity, as expected by one of ordinary skill in the art in making the measurement and exercising a level of care commensurate with the objective of measurement and precisions of the measuring equipment and any quantities higher than that. In certain embodiments, the term “at least about” includes the recited number minus 10% and any quantity that is higher such that “at least about 10” would include 9 and anything greater than 9. This term can also be expressed as “about 10 or more.” Similarly, the term “less than about” typically includes the recited number plus 10% and any quantity that is lower such that “less than about 10” would include 11 and anything less than 11. This term can also be expressed as “about 10 or less.”

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to illuminate certain materials and methods and does not pose a limitation on scope. No language in the specification should be construed as indicating any non-claimed element as essential to the practice of the disclosed materials and methods.

Although the operations of the methods herein are shown and described in a particular order, the order of the operations of each method may be altered so that certain operations may be performed in an inverse order or so that certain operation may be performed, at least in part, concurrently with other operations. In another embodiment, instructions or sub-operations of distinct operations may be in an intermittent and/or alternating manner.

It is to be understood that the above description is intended to be illustrative, and not restrictive. Many other embodiments will be apparent to those of skill in the art upon reading and understanding the above description. The scope of the disclosure should, therefore, be determined with reference to the appended claims, along with the full scope of equivalents to which such claims are entitled.

What is claimed is:

1. A dissolvable solid foam composition, comprising:
a soluble polymer; and
a surfactant,
wherein the composition is a closed cell foam.

2. The composition of claim 1, wherein the soluble polymer comprises polyvinyl pyrrolidone, polyvinyl alcohol, cellulose acetate, modified wood pulp, modified paper pulp or a combination of any two or more of the foregoing.

3. The composition of claim 1 or 2, wherein the soluble polymer is in an amount of about 1% by weight to about 99% by weight of the total weight of the composition.

4. The composition according to any preceding claim, wherein the surfactant comprises a saponin, an alcohol ethoxylate, an oil ethoxylate, lauramine oxide, a lauryl alcohol ethoxylate, or a combination of any two or more of the foregoing.

5. The composition according to any preceding claim, wherein the surfactant is in an amount of about 1% by weight to about 99% by weight of the total weight of the composition, or less than 23% by weight of the total weight of the composition, or greater than 75% by weight of the total weight of the composition.

6. The composition according to any preceding claim, further comprising an exfoliant powder, optionally wherein the exfoliant powder comprises rough grains, seed meal, corn meal, coffee grounds, sand, activated charcoal, or a combination of any two or more of the foregoing.

7. The composition according to any preceding claim, further comprising an exfoliant powder, optionally wherein the exfoliant powder is in an amount of greater than 0% by weight to about 5% by weight of the total weight of the composition.

8. The composition according to any preceding claim, further comprising an exfoliant powder, optionally wherein the exfoliant powder comprises particles, granules, pellets, extrudates, beads or a combination of any two or more of the foregoing.

9. The composition according to any preceding claim, further comprising an exfoliant powder, optionally wherein the exfoliant powder comprises a particle size distribution of less than about 500 μm or a mean particle size of about 1 μm to about 500 μm.

10. The composition according to any preceding claim, further comprising an additive comprising a fragrance, a moisturizer, a vitamin or a combination of any two or more of the foregoing.

11. The composition of claim 10, wherein the fragrance comprises a perfume, an essential oil, an extract, an absolute, a resinoid, a concentrate, eucalyptus globulus leaf oil, prunus amygdalus dulcis oil, lavender oil, rosemary oil, vanilla oil, tea tree oil, peppermint oil, spearmint oil, sweet orange oil, lemon oil, or a combination of any two or more of the foregoing.

12. The composition of claim 10 or 11, wherein the fragrance is in an amount of less than about 10% by weight or less than about 1% by weight based on the total weight of the composition.

13. The composition according to any one of claims 10 to 12, wherein the moisturizer comprises sodium hyaluronate, aloe, vitamin E oil, a tocopherol, a tocotrienol, shea oil, olive oil, jojoba oil, sesame oil, rosehip seed oil, apricot kernel oil, coconut oil, sweet almond oil, sandalwood oil, jasmine oil, juniper oil, vitamin A, a retinol ester, a retinal ester, a retinyl ester, niacin, L-ascorbic acid, zinc, or a combination of any two or more of the foregoing.

14. The composition according to any one of claims 10 or 13, wherein the moisturizer is in an amount of about 1% by weight to about 50% by weight based on the total weight of the composition.

15. The composition according to any one of claims 10 to 14, wherein the vitamin comprises vitamin E, vitamin A, vitamin B3, vitamin C or a combination of any two or more of the foregoing.

16. The composition according to any one of claims 10 to 15, wherein the vitamin is in an amount of less than about 10% by weight, or any individual value or sub-range therein.

17. The composition according to any preceding claim, wherein the composition further comprises pinholes.

18. A biodegradable solid foam composition, comprising:
a soluble polymer comprising polyvinyl alcohol; and
a surfactant comprising an ethoxylate;
wherein the solid foam composition biodegrades in less than about 1 year.

19. The composition of claim 18, wherein the soluble polymer further comprises polyvinyl pyrrolidone, cellulose acetate, modified wood pulp, modified paper pulp or a combination of any two or more of the foregoing.

20. The composition of claim 18 or 19, wherein the soluble polymer is in an amount of about 1% by weight to about 99% by weight of the total weight of the composition.

21. The composition according to any one of claims 18 to 20, wherein the surfactant further comprises a saponin, lauramine oxide, or a combination of any two or more of the foregoing.

22. The composition according to any one of claims 18 to 21, wherein the ethoxylate comprises an alcohol ethoxylate, an oil ethoxylate, a lauryl alcohol ethoxylate, or a combination of any two or more of the foregoing.

23. The composition according to any one of claims 18 to 22, wherein the surfactant is in an amount of about 1% by weight to about 99% by weight of the total weight of the composition, or less than 23% by weight of the total weight of the composition, or greater than 75% by weight of the total weight of the composition.

24. The composition according to any one of claims 18 to 23, further comprising an exfoliant powder, optionally wherein the exfoliant powder comprises rough grains, seed meal, corn meal, coffee grounds, sand, activated charcoal, or a combination of any two or more of the foregoing.

25. The composition according to any one of claims 18 to 24, further comprising an exfoliant powder, optionally wherein the exfoliant powder is in an amount of greater than 0% by weight to about 5% by weight of the total weight of the composition.

26. The composition according to any one of claims 18 to 25, further comprising an exfoliant powder, optionally wherein the exfoliant powder comprises particles, granules, pellets, extrudates, beads or a combination of any two or more of the foregoing.

27. The composition according to any one of claims 18 to 26, further comprising an exfoliant powder, optionally wherein the exfoliant powder comprises a particle size distribution of less than about 500 μm or a mean particle size of about 1 μm to about 500 μm.

28. The composition according to any one of claims 18 to 27, further comprising an additive comprising a fragrance, a moisturizer, a vitamin or a combination of any two or more of the foregoing.

29. The composition of claim 28, wherein the fragrance comprises a perfume, an essential oil, an extract, an absolute, a resinoid, a concentrate, eucalyptus globulus leaf oil, prunus amygdalus dulcis oil, lavender oil, rosemary oil, vanilla oil, tea tree oil, peppermint oil, spearmint oil, sweet orange oil, lemon oil, or a combination of any two or more of the foregoing.

30. The composition of claim 28 or 29, wherein the fragrance is in an amount of less than about 10% by weight or less than about 1% by weight based on the total weight of the composition.

31. The composition according to any one of claims 28 to 30, wherein the moisturizer comprises sodium hyaluronate, aloe, vitamin E oil, a tocopherol, a tocotrienol, shea oil, olive oil, jojoba oil, sesame oil, rosehip seed oil, apricot kernel oil, coconut oil, sweet almond oil, sandalwood oil, jasmine oil, juniper oil, vitamin A, a retinol ester, a retinal ester, a retinyl ester, niacin, L-ascorbic acid, zinc, or a combination of any two or more of the foregoing.

32. The composition according to any one of claims 28 or 31, wherein the moisturizer is in an amount of about 1% by weight to about 50% by weight based on the total weight of the composition.

33. The composition according to any one of claims 28 to 32, wherein the vitamin comprises vitamin E, vitamin A, vitamin B3, vitamin C or a combination of any two or more of the foregoing.

34. The composition according to any one of claims 28 to 33, wherein the vitamin is in an amount of less than about 10% by weight, or any individual value or sub-range therein.

35. The composition according to any one of claims 18 to 34, wherein the composition further comprises pinholes.

36. The composition of claim 18, wherein the solid foam is a closed cell foam.

37. A personal care product, comprising:
a dissolvable solid foam composition according to any one of claims 1 to 36; and
a sachet overlaying the dissolvable solid foam composition, wherein the sachet comprises a soluble polymer.

38. A personal care product, comprising:
a dissolvable solid foam composition according to any one of claims 1 to 36, wherein the personal care product is selected from the group consisting of a body soap, a shaving cream, a moisturizer and a sunscreen.

39. A kit, comprising:
a plurality of pieces of a dissolvable solid foam composition according to any one of claims 1 to 36;
a plurality of sachets, each sachet comprising a soluble polymer and configured to overlay at least one of the plurality of pieces; and
instructions to:
contact at least one of the plurality of pieces and a corresponding sachet with water to form a cleansing composition within about 5 sec to about 60 sec;
apply the cleansing composition to skin or hair for at least 30 sec; and
rinse the cleansing composition with water.
